SEQUENCE LISTING (12 sequences)

SEQ ID NO: 1 - DNA, 993 bp, Synthetic construct
  source 1..993: mol_type = other DNA
atggctttctctcaagataccgcggaaatcgtttacacccacgacaccggtctggactatatcacctactctgactacgaactggacccgcagaacccgctggctggtggtgctgcttggatcgaaggtgctttcgttccgccgtctgaagctcgtatctgcatcttcgaccagggttacattcactctgacgttacctacaccgttttccacgtttggaacggtaacgctttccgtctggacgaccacatcgaacgtctgttctctaacgctgaatctattcgtatcatcccgccgctgaccaaagacgaagttaaagaaatcgctctggaactggttgctaaaaccgaactgcgtgaagcattcgtttctgtttctatcacccgtggttactcttctaccccatgggaacgtgacatcaccaaacatcgtccgcaggtttacatgtacgctgttccgtaccagtggatcgtaccgtttgacggcatccgtaacggtgttcacgctatggttgctcagtctgttcgtcgtactccgcgttggtctatcgacccgcagattaaaaacttccagtggggtgacctgatccgtgcaattcaggaaacgcacgaccgtggtttcgaactgccgctgctgctggacgcagacggtttgctggctgaaggtccaggtttcaacgttgttgttatcaaagacgatgttgttcgttctccgggtcgtgctgctctgccgggtatcacccgtaaaaccgttctggaaatcgctgaatctctgggtcacgaagctatcctggctgacatcaccctggctgaactgctggacgctgacgaagttctgggttgcactaccgctggtggtgtttggccgttcgtttctgttgacggtaacccgatctctgacggtgttccgggtccggttacccagtctatcatccgtcgttactgggaactgaacgttgaatcttcttctctgctgaccccggtacagtactaa

SEQ ID NO: 2 - protein, 330 aa, Synthetic construct
MAFSQDTAEIVYTHDTGLDYITYSDYELDPQNPLAGGAAWIEGAFVPPSEARICIFDQGYIHSDVTYTVFHVWNGNAFRLDDHIERLFSNAESIRIIPPLTKDEVKEIALELVAKTELREAFVSVSITRGYSSTPWERDITKHRPQVYMYAVPYQWIVPFDGIRNGVHAMVAQSVRRTPRWSIDPQIKNFQWGDLIRAIQETHDRGFELPLLLDADGLLAEGPGFNVVVIKDDVVRSPGRAALPGITRKTVLEIAESLGHEAILADITLAELLDADEVLGCTTAGGVWPFVSVDGNPISDGVPGPVTQSIIRRYWELNVESSSLLTPVQY

SEQ ID NO: 3 - DNA, 993 bp, Synthetic construct
  source 1..993: mol_type = other DNA
atggctttctctgctgacacccctgaaatcgtttacacccacgacaccggtctggactatatcacctactctgactacgaactggacccggctaacccgctggctggtggtgctgcttggatcgaaggtgctttcgttccgccgtctgaagctcgtatctctatcttcgaccagggttacctgcactctgacgttacctacaccgttttccacgtttggaacggtaacgctttccgtctggacgaccacatcgaacgtctgttctctaacgctgaatctatgcgtatcatcccgccgctgacccaggacgaagttaaagaaatcgctctggaactggttgctaaaaccgaactgcgtgaagcattcgtttctgtttctatcacccgtggttactcttctaccccatatgaacgtgacatcaccaaacatcgtccgcaggtttacatgtacgctgttccgtaccagtggatcgtaccgtttgaccgcatccgtgacggtgttcacgctatggttgctcagtctgttcgtcgtactccgcgttcttctatcgacccgcaggttaaaaacttccagtggggtgacctgatccgtgcagttcaggaaacccacgaccgtggtttcgaagctccgctgctgctggacggtgacggtctgctggctgaaggttctggtttcaacgttgttgttatcaaagacgatgttgttcgttctccgggtcgtgctgctctgccgggtatcacccgtaaaaccgttctggaaatcgctgaatctctgggtcacgaagctatcctggctgacatcaccctggctgaactgctggacgctgacgaagttctgggttgcactaccgctggtggtgtttggccgttcgtttctgttgacggtaacccgatctctgacggtgttccgggtccggttacccagtctatcatccgtcgttactgggaactgaacgttgaatcttcttctctgctgaccccggtacagtactaa

SEQ ID NO: 4 - protein, 330 aa, Synthetic construct
MAFSADTPEIVYTHDTGLDYITYSDYELDPANPLAGGAAWIEGAFVPPSEARISIFDQGYLHSDVTYTVFHVWNGNAFRLDDHIERLFSNAESMRIIPPLTQDEVKEIALELVAKTELREAFVSVSITRGYSSTPYERDITKHRPQVYMYAVPYQWIVPFDRIRDGVHAMVAQSVRRTPRSSIDPQVKNFQWGDLIRAVQETHDRGFEAPLLLDGDGLLAEGSGFNVVVIKDDVVRSPGRAALPGITRKTVLEIAESLGHEAILADITLAELLDADEVLGCTTAGGVWPFVSVDGNPISDGVPGPVTQSIIRRYWELNVESSSLLTPVQY

SEQ ID NO: 5 - DNA, 993 bp, Synthetic construct
  source 1..993: mol_type = other DNA
atggctttctctgctgacacccctgaaatcgtttacacccacgacaccggtctggactatatcacctactctgactacgaactggacccggctaacccgctggctggtggtgctgcttggatcgaaggtgctttcgttccgccgtctgaagctcgtatctctatcttcgaccagggttacctgcactctgacgttacctacaccgttttccacgtttggaacggtaacgctttccgtctggacgaccacatcgaacgtctgttctctaacgctgaatctatgcgtatcatcccgccgctgacccaggacgaagttaaagaaatcgctctggaactggttgctaaaaccgaactgcgtgaagcattcgtttctgtttctatcacccgtggttactcttctaccccatatgaacgtgacatcaccaaacatcgtccgcaggtttacatgtacgctgttccgtaccagtggatcgtaccgtttgaccgcatccgtgacggtgttcacgctatggttgctcagtctgttcgtcgtactccgcgttcttctatcgacccgcaggttaaaaacttccagtggggtgacctgatccgtgcagttcaggaaacccacgaccgtggtttcgaagctccgctgctgctggacggtgacggtctgctggctgaaggtccaggtttcaacgttgttgttatcaaagacgatgttgttcgttctccgggtcgtgctgctctgccgggtatcacccgtaaaaccgttctggaaatcgctgaatctctgggtcacgaagctatcctggctgacatcaccctggctgaactgctggacgctgacgaagttctgggttgcactaccgctggtggtgtttggccgttcgtttctgttgacggtaacccgatctctgacggtgttccgggtccggttacccagtctatcatccgtcgttactgggaactgaacgttgaatcttcttctctgctgaccccggtacagtactaa

SEQ ID NO: 6 - protein, 330 aa, Synthetic construct
MAFSADTPEIVYTHDTGLDYITYSDYELDPANPLAGGAAWIEGAFVPPSEARISIFDQGYLHSDVTYTVFHVWNGNAFRLDDHIERLFSNAESMRIIPPLTQDEVKEIALELVAKTELREAFVSVSITRGYSSTPYERDITKHRPQVYMYAVPYQWIVPFDRIRDGVHAMVAQSVRRTPRSSIDPQVKNFQWGDLIRAVQETHDRGFEAPLLLDGDGLLAEGPGFNVVVIKDDVVRSPGRAALPGITRKTVLEIAESLGHEAILADITLAELLDADEVLGCTTAGGVWPFVSVDGNPISDGVPGPVTQSIIRRYWELNVESSSLLTPVQY

SEQ ID NO: 7 - DNA, 993 bp, Synthetic construct
  source 1..993: mol_type = other DNA
atggctttctctgctgacacccctgaaatcgtttacacccacgacaccggtctggactatatcacctactctgactacgaactggacccggctaacccgctggctggtggtgctgcttggatcgaaggtgctttcgttccgccgtctgaagctcgtatctctatcttcgaccagggttacattcactctgacgttacctacaccgttttccacgtttggaacggtaacgctttccgtctggacgaccacatcgaacgtctgttctctaacgctgaatctattcgtatcatcccgccgctgacccaggacgaagttaaagaaatcgctctggaactggttgctaaaaccgaactgcgtgaagcattcgtttctgtttctatcacccgtggttactcttctaccccatgggaacgtgacatcaccaaacatcgtccgcaggtttacatgtacgctgttccgtaccagtggatcgtaccgtttgaccgcatccgtgacggtgttcacgctatggttgctcagtctgttcgtcgtactccgcgttcttctatcgacccgcagattaaaaacttccagtggggtgacctgatccgtgcagttcaggaaacccacgaccgtggtttcgaactgccgctgctgctggacggtgacggtctgctggctgaaggtccaggtttcaacgttgttgttatcaaagacgatgttgttcgttctccgggtcgtgctgctctgccgggtatcacccgtaaaaccgttctggaaatcgctgaatctctgggtcacgaagctatcctggctgacatcaccctggctgaactgctggacgctgacgaagttctgggttgcactaccgctggtggtgtttggccgttcgtttctgttgacggtaacccgatctctgacggtgttccgggtccggttacccagtctatcatccgtcgttactgggaactgaacgttgaatcttcttctctgctgaccccggtacagtactaa

SEQ ID NO: 8 - protein, 330 aa, Synthetic construct
MAFSADTPEIVYTHDTGLDYITYSDYELDPANPLAGGAAWIEGAFVPPSEARISIFDQGYIHSDVTYTVFHVWNGNAFRLDDHIERLFSNAESIRIIPPLTQDEVKEIALELVAKTELREAFVSVSITRGYSSTPWERDITKHRPQVYMYAVPYQWIVPFDRIRDGVHAMVAQSVRRTPRSSIDPQIKNFQWGDLIRAVQETHDRGFELPLLLDGDGLLAEGPGFNVVVIKDDVVRSPGRAALPGITRKTVLEIAESLGHEAILADITLAELLDADEVLGCTTAGGVWPFVSVDGNPISDGVPGPVTQSIIRRYWELNVESSSLLTPVQY

SEQ ID NO: 9 - DNA, 993 bp, Synthetic construct
  source 1..993: mol_type = other DNA
atggctttctctcaagataccgcggaaatcgtttacacccacgacaccggtctggactatatcacctactctgactacgaactggacccgcagaacccgctggctggtggtgctgcttggatcgaaggtgctttcgttccgccgtctgaagctcgtatctgcatcttcgaccagggttacattcactctgacgttacctacaccgttttccacgtttggaacggtaacgctttccgtctggacgaccacatcgaacgtctgttctctaacgctgaatctattcgtatcatcccgccgctgaccaaagacgaagttaaagaaatcgctctggaactggttgctaaaaccgaactgcgtgaagcattcgtttctgtttctatcacccgtggttactcttctaccccatgggaacgtgacatcaccaaacatcgtccgcaggtttacatgtacgctgttccgtaccagtggatcgtaccgtttgacggcatccgtgacggtgttcacgctatggttgctcagtctgttcgtcgtactccgcgttggtctatcgacccgcagattaaaaacttccagtggggtgacctgatccgtgcagttcaggaaacccacgaccgtggtttcgaactgccgctgctgctggacggtgacggtctgctggctgaaggtccaggtttcaacgttgttgttatcaaagacgatgttgttcgttctccgggtcgtgctgctctgccgggtatcacccgtaaaaccgttctggaaatcgctgaatctctgggtcacgaagctatcctggctgacatcaccctggctgaactgctggacgctgacgaagttctgggttgcactaccgctggtggtgtttggccgttcgtttctgttgacggtaacccgatctctgacggtgttccgggtccggttacccagtctatcatccgtcgttactgggaactgaacgttgaatcttcttctctgctgaccccggtacagtactaa

SEQ ID NO: 10 - protein, 330 aa, Synthetic construct
MAFSQDTAEIVYTHDTGLDYITYSDYELDPQNPLAGGAAWIEGAFVPPSEARICIFDQGYIHSDVTYTVFHVWNGNAFRLDDHIERLFSNAESIRIIPPLTKDEVKEIALELVAKTELREAFVSVSITRGYSSTPWERDITKHRPQVYMYAVPYQWIVPFDGIRDGVHAMVAQSVRRTPRWSIDPQIKNFQWGDLIRAVQETHDRGFELPLLLDGDGLLAEGPGFNVVVIKDDVVRSPGRAALPGITRKTVLEIAESLGHEAILADITLAELLDADEVLGCTTAGGVWPFVSVDGNPISDGVPGPVTQSIIRRYWELNVESSSLLTPVQY

SEQ ID NO: 11 - DNA, 993 bp, Synthetic construct
  source 1..993: mol_type = other DNA
atggctttctctcaagataccgcggaaatcgtttacacccacgacaccggtctggactatatcacctactctgactacgaactggacccgcagaacccgctggctggtggtgctgcttggatcgaaggtgctttcgttccgccgtctgaagctcgtatctgcatcttcgaccagggttacattcactctgacgttacctacaccgttttccacgtttggaacggtaacgctttccgtctggacgaccacatcgaacgtctgttctctaacgctgaatctattcgtatcatcccgccgctgaccaaagacgaagttaaagaaatcgctctggaactggttgctaaaaccgaactgcgtgaagcattcgtttctgtttctatcacccgtggttactcttctaccccatgggaacgtgacatcaccaaacatcgtccgcaggtttacatgtacgctgttccgtaccagtggatcgtaccgtttgacggcatccgtgacggtgttcacgctatggttgctcagtctgttcgtcgtactccgcgttggtctatcgacccgcagattaaaaacttccagtggggtgacctgatccgtgcaattcaggaaacccacgaccgtggtttcgaactgccgctgctgctggacggtgacggtctgctggctgaaggtccaggtttcaacgttgttgttatcaaagacgatgttgttcgttctccgggtcgtgctgctctgccgggtatcacccgtaaaaccgttctggaaatcgctgaatctctgggtcacgaagctatcctggctgacatcaccctggctgaactgctggacgctgacgaagttctgggttgcactaccgctggtggtgtttggccgttcgtttctgttgacggtaacccgatctctgacggtgttccgggtccggttacccagtctatcatccgtcgttactgggaactgaacgttgaatcttcttctctgctgaccccggtacagtactaa

SEQ ID NO: 12 - protein, 330 aa, Synthetic construct
MAFSQDTAEIVYTHDTGLDYITYSDYELDPQNPLAGGAAWIEGAFVPPSEARICIFDQGYIHSDVTYTVFHVWNGNAFRLDDHIERLFSNAESIRIIPPLTKDEVKEIALELVAKTELREAFVSVSITRGYSSTPWERDITKHRPQVYMYAVPYQWIVPFDGIRDGVHAMVAQSVRRTPRWSIDPQIKNFQWGDLIRAIQETHDRGFELPLLLDGDGLLAEGPGFNVVVIKDDVVRSPGRAALPGITRKTVLEIAESLGHEAILADITLAELLDADEVLGCTTAGGVWPFVSVDGNPISDGVPGPVTQSIIRRYWELNVESSSLLTPVQY

(19) United States
(12) Patent Application Publication
(10) Pub. No.: US 2025/0034607 A1
(43) Pub. Date: Jan. 30, 2025

(54) Process of Synthesizing (R)-3-aminobutan-1-ol

(71) Applicant: ViiV Healthcare Company, Wilmington, DE (US)

(72) Inventors: Mark BOBKO, Collegeville, PA (US); Douglas FUERST, Collegeville, PA (US); Christopher MORGAN, Collegeville, PA (US)

(21) Appl. No.: 18/715,393

(22) PCT Filed: Nov. 29, 2022
(86) PCT No.: PCT/US2022/080557

§ 371 Date: May 31, 2024

Related U.S. Application Data
(60) Provisional application No. 63/285,606, filed on Dec. 3, 2021.
(60) Provisional application No. 63/365,961, filed on Jun. 7, 2022.

(51) Int. Cl. C12P 13/00; C12N 9/10

(52) U.S. Cl. CPC C12P 13/001; C12N 9/1096

(57) ABSTRACT

The present invention relates to an aminotransaminase useful in the synthesis of (R)-3-aminobutan-1-ol (RABO). The present invention also provides a process of preparing (R)-3-aminobutan-1-ol (RABO) with the disclosed aminotransaminase.

### FIELD OF THE INVENTION

The present disclosure relates to processes of synthesizing (R)-3-aminobutan-1-ol (RABO) and to polypeptides having aminotransaminase activity.

### BACKGROUND TO THE INVENTION

(4R,12aS)-9-{[(2,4-difluorophenyl)methyl]carbamoyl}-4-methyl-6,8-dioxo-3,4,6,8,12,12a-hexahydro-2H-pyrido[1′,2′:4,5]pyrazino[2,1-b][1,3]oxazin-7-olate, known as Dolutegravir (DTG), is an integrase strand transfer inhibitor (INSTI) used in the form of a sodium salt in combination with other medications to treat HIV. See U.S. Pat. No. 8,129,385.

DTG contains an S hemiaminal stereocenter that is important for protein binding and potency against common HIV mutants. According to at least one method of making DTG, the favorable stereochemistry of DTG is obtained by using an isomeric (R)-3-aminobutan-1-ol intermediate in a final ring closing step (Johns et al., J. Med. Chem. 2013, 56, 5901-5916). (R)-3-aminobutan-1-ol (RABO), however, is relatively expensive and can be a major cost driver for DTG production—accounting for nearly 30% of the overall cost. The synthesis of small chiral alcohols is challenging for many reasons including purification complications because of their low boiling points (Medicines for All Initiative, Process Development Report, Synthesis of (R)-3-aminobutan-1-ol, Nov. 18, 2019).

Transaminases or “aminotransaminases” are polypeptides having an enzymatic capability of transferring an amino group (—NH2), from a primary amine of an amine donor compound to the carbonyl group (C═O) of an amine acceptor compound. Aminotransaminase reactions may be used in the manufacturing process of dolutegravir and more specifically, the RABO intermediate. See WO2018/020380. However, these current aminotransaminase reactions still result in inefficient RABO production.

As such, there is a need to identify processes of synthesizing RABO and aminotransaminases useful in the synthesis of RABO that increase production efficiency and ultimately reduce the overall product costs of specific antiretroviral treatments for HIV.

### SUMMARY OF THE INVENTION

According to a first aspect of the invention, there is provided a process for preparing a compound of structural Formula (I):

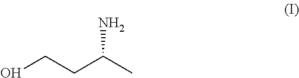

the process comprising the step of reacting 4-hydroxy-2-butanone with an amine donor in the presence of an aminotransaminase, wherein the aminotransaminase comprises an amino acid sequence that is at least 80% identical to SEQ ID NO. 4 and wherein the aminotransaminase comprises the following mutations relative to SEQ ID NO. 4: X5Q, X8A, X31Q, X54C, X61I, X94I, X102K, X136W, X162G, X181W, X187I, X199I, X209L and X223P.

According to a second aspect of the invention, there is provided an aminotransaminase comprising an amino acid sequence that is at least 80% identical to SEQ ID NO. 4 and wherein the aminotransaminase comprises the following mutations relative to SEQ ID NO. 4: X5Q, X8A, X31Q, X54C, X61I, X94I, X102K, X136W, X162G, X181W, X187I, X199I, X209L and X223P.

According to a third aspect of the invention, there is provided an aminotransaminase comprising an amino acid sequence of SEQ ID NO. 2.

According to a further aspect of the invention, there is provided an aminotransaminase consisting of the amino acid sequence of SEQ ID NO. 2.

According to a further aspect of the invention, there is provided a polynucleotide encoding an aminotransaminase of the present invention.

According to a final aspect of the invention, there is provided use of an aminotransaminase of the present invention, for the manufacture of a compound of structural Formula (I):

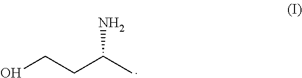

The present invention is advantageous in a number of respects. Specifically, the method of making RABO with the disclosed aminotransaminases, having improved properties such as increased activity and thermostability, may decrease reaction time, improve yield/conversion rate and/or stereochemistry selectivity, reduce production costs and increase accessibility to those in need of antiretroviral therapy.

### DETAILED DESCRIPTION OF THE INVENTION

### Definitions

As used herein, the terms “protein,” “polypeptide” and “peptide” are used interchangeably herein to denote a polymer of at least two amino acids covalently linked by an amide bond, regardless of length or post-translational modification (e.g., glycosylation, phosphorylation, lipidation, myristylation, ubiquitination, etc.). Included within this definition are D- and L-amino acids and mixtures of D- and L-amino acids.

As used herein, the terms “aminotransferase,” “transaminase” and “aminotransaminase” are used interchangeably and refer to a polypeptide having an enzymatic capability of transferring an amino group (—NH₂), a pair of electrons and a proton from the primary amine of an amine donor compound to the carbonyl group (C═O) of an amine acceptor compound, thereby converting the amine donor compound into its corresponding carbonyl compound and the carbonyl acceptor compound into its corresponding primary amine compound (See e.g., Scheme 1).

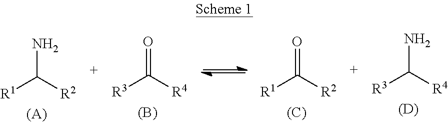

Aminotransaminases, as used herein, include naturally occurring (wild type) aminotransaminases as well as non-naturally occurring engineered polypeptides generated by human manipulation.

The phrase “residue difference” or “amino acid difference” refers to a change in the residue at a specified position of a polypeptide sequence when compared to a reference sequence. The polypeptide sequence position at which a particular amino acid or amino acid change is present is sometimes described herein as “Xn”, or “position n”, where n refers to the residue position with respect to the reference sequence. For example, a residue difference at position X8, where the reference sequence has a serine, refers to a change of the residue at position X8 to any residue other than serine. As disclosed herein, an aminotransferase of the present invention can include one or more residue differences relative to a reference sequence, where multiple residue differences typically are indicated by a list of the specified positions where changes are made relative to the reference sequence. A specific substitution mutation, which is a replacement of the specific residue in a reference sequence with a different specified residue may be denoted by the conventional notation X(number)Y”, where X is the single letter identifier of the residue in the reference sequence, “number” is the residue position in the reference sequence and Y is the single letter identifier of the residue substitution in the engineered sequence. Thus, for example, “the mutation X5Q relative to SEQ ID NO. 4” means that the 5^{th} amino acid counting from the N-terminal of SEQ ID NO. 4 is changed to Q.

As used herein, the term “amine donor” or “amino donor” are used interchangeably to refer to a compound containing an amino group that is capable of donating an amino group to an acceptor carbonyl compound (i.e. an amino group acceptor), thereby becoming a carbonyl by-product. In one embodiment, the amine donor used in the present invention have the general structural formula:

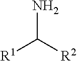

in which each of R¹ and R², when taken independently, is an alkyl, an alkyl aryl group, an aryl group which is unsubstituted or substituted with one or more enzymatically non-inhibiting groups (see (A), Scheme 1). R¹ can be the same or different from R² in structure or chirality. Typical amino group donors include chiral and achiral amino acids and chiral and achiral amines.

As used herein, the term “carbonyl by-product” refers to the carbonyl compound formed from the amino group donor when the amino group on the amino group donor is transferred to the amino group acceptor in a transamination reaction. The carbonyl by-product has the general structural formula:

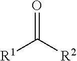

wherein R¹ and R² are defined above for the amino group donor (see (C), Scheme 1).

As used herein, the terms “amino acceptor,” “amine acceptor,” “keto substrate,” “substrate” or “substrate compound” are used interchangeably to refer to a carbonyl group containing a compound that accepts the amino group from an amino group donor in a reaction mediated by an aminotransaminase (see (B), Scheme 1).

As used herein, the term “cofactor” refers to a non-protein compound that operates in combination with an enzyme in catalysing a reaction. As used here, “cofactor” is intended to encompass the vitamin B₆ family compounds PLP, PN, PL, PM, PNP and PMP, which are sometimes referred to as coenzymes.

As used herein, the terms “pyridoxal-phosphate,” “PLP,” “pyridoxal-5′-phosphate,” “PYP,” and “P5P” are used interchangeably herein to refer to the compound that acts as a cofactor in aminotransaminase reactions. In some embodiments, pyridoxal phosphate is defined by the structure 1-(4′-formyl-3′-hydroxy-2 i-methyl 5′-pyridyl) methoxyphosphonic acid, CAS number [54-47 R2 7]. Pyridoxal-5′-phosphate can be produced in vivo by phosphorylation and oxidation of pyridoxal (also known as Vitamin B.). In transamination reactions using aminotransaminase enzymes, the amine group of the amino donor is transferred to the cofactor to produce a keto by-product, while pyridoxal-5′-phosphate is converted to pyridoxamine phosphate. Pyridoxal-5′-phosphate regenerated by reaction with a different keto compound (the amino acceptor). The transfer of the amine group from pyridoxamine phosphate to the amino acceptor produces an amine and regenerates the cofactor. In some embodiments, the pyridoxal-5′phosphate can be replaced by other members of the vitamin B₆ family including pyridoxine (PN), pyridoxal (PL), pyridoxamine (PM) and their phosphorylated counterparts; pyridoxine phosphate (PNP) and pyridoxamine phosphate (PMP).

As used herein, the term “enzyme” means a series of proteins that act as biological catalysts.

As used herein, the term “biological catalyst” means a linear polypeptide that accelerates chemical reactions or chemical transformations of organic compounds.

As used herein, “coding sequence” refers to the portion of a nucleic acid (e.g., a gene) that encodes an amino acid sequence of a protein.

As used herein, “naturally-occurring” or “wild-type” refers to the form found in nature. For example, a naturally occurring or wild-type polypeptide or polynucleotide sequence is a sequence present in an organism that can be isolated from a source in nature and which has not been intentionally modified by human manipulation.

As used herein, “recombinant” or “engineered” or “non-naturally occurring” when used with reference to, e.g., a cell, nucleic acid, or polypeptide, refers to a material, or a material corresponding to the natural or native form of the material, that has been modified in a manner that would not otherwise exist in nature, or is identical thereto but produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

“Percent identity” or “% identity” between a query nucleic acid sequence and a subject nucleic acid sequence is the “identities” value, expressed as a percentage, that is calculated using a suitable algorithm (e.g. BLASTN, FASTA, Needleman-Wunsch, Smith-Waterman, LALIGN, or GenePAST/KERR) or software (e.g. DNASTAR Lasergene, GenomeQuest, EMBOSS needle or EMBOSS infoalign), over the entire length of the query sequence after a pair-wise global sequence alignment has been performed using a suitable algorithm (e.g. Needleman-Wunsch or GenePAST/KERR) or software (e.g. DNASTAR Lasergene or GenePAST/KERR). Importantly, a query nucleic acid sequence may be described by a nucleic acid sequence disclosed herein, in particular in one or more of the claims.

“Percent identity” or “% identity” between a query amino acid sequence and a subject amino acid sequence is the “identities” value, expressed as a percentage, that is calculated using a suitable algorithm (e.g. BLASTP, FASTA, Needleman-Wunsch, Smith-Waterman, LALIGN, or GenePAST/KERR) or software (e.g. DNASTAR Lasergene, GenomeQuest, EMBOSS needle or EMBOSS infoalign), over the entire length of the query sequence after a pair-wise global sequence alignment has been performed using a suitable algorithm (e.g. Needleman-Wunsch or GenePAST/KERR) or software (e.g. DNASTAR Lasergene or GenePAST/KERR). Importantly, a query amino acid sequence may be described by an amino acid sequence disclosed herein, in particular in one or more of the claims.

The query sequence may be 100% identical to the subject sequence, or it may include up to a certain integer number of amino acid alterations as compared to the subject sequence such that the % identity is less than 100%. For example, the query sequence is at least 50, 60, 70, 75, 80, 85, 90, 95, 96, 97, 98, or 99% identical to the subject sequence. Such alterations include at least one amino acid deletion, substitution (including conservative and non-conservative substitution) or insertion and wherein said alterations may occur at the amino- or carboxy-terminal positions of the query sequence or anywhere between those terminal positions, interspersed either individually among the amino acids in the query sequence or in one or more contiguous groups within the query sequence.

As used herein, “reference sequence” refers to a defined sequence used as a basis for a sequence comparison. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length or the full length of the nucleic acid or polypeptide. Since two polynucleotides or polypeptides may each (1) comprise a sequence (i.e., a portion of the complete sequence) that is similar between the two sequences and (2) may further comprise a sequence that is divergent between the two sequences, sequence comparisons between two (or more) polynucleotides or polypeptide are typically performed by comparing sequences of the two polynucleotides or polypeptides over a “comparison window” to identify and compare local regions of sequence similarity. In some embodiments, a “reference sequence” can be based on a primary amino acid sequence, where the reference sequence is a sequence that can have one or more changes in the primary sequence.

As used herein, “improved enzyme property” refers to an aminotransaminase polypeptide that exhibits an improvement in any enzyme property as compared to a reference aminotransaminase, such as the wild-type aminotransaminase enzyme or another improved engineered aminotransaminase. Enzyme properties for which improvement is desirable include, but are not limited to, enzymatic activity (which can be expressed in terms of percent conversion of the substrate), thermostability, solvent stability, pH activity profile, cofactor requirements, refractoriness to inhibitors (e.g., substrate or product inhibition), stereospecificity and stereoselectivity (including enantioselectivity).

As used herein, “increased or greater enzymatic activity” refers to improved activity of the engineered aminotransaminase polypeptides, which can be represented by an increase in specific activity (e.g., product produced/time/weight protein) or an increase in percent conversion of the substrate to the product (e.g., percent conversion of amount of substrate to product in a specified time period using a specified amount of aminotransaminase) as compared to the reference aminotransaminase enzyme. Exemplary methods to determine enzyme activity are provided in the Examples. Any property relating to enzyme activity may be affected, including the classical enzyme properties of Km, Vmax or kcar, changes of which can lead to increased enzymatic activity. Improvements in enzyme activity can be from about 1.1 fold improvement over parent (FIOP) the enzymatic activity of the corresponding wild-type aminotransaminase enzyme, to as much as 2 FIOP, 5 FIOP, 10 FIOP, 20 FIOP, 25 FIOP, 50 FIOP, 75 FIOP, 100 FIOP, or more enzymatic activity than the naturally occurring aminotransaminase or another engineered aminotransaminase from which the aminotransaminase polypeptides were derived. Aminotransaminase activity can be measured by any one of standard assays, such as by monitoring changes in spectrophotometric properties of reactants or products. In some embodiments, the amount of products produced can be measured by known assays using High-Performance Liquid Chromatography (HPLC) separation combined with UV absorbance or fluorescent detection following o-Phthaldialdehyde (OPA) derivatization. Comparisons of enzyme activities are made using a defined preparation of enzyme, a defined assay under a set condition and one or more defined substrates, as further described in detail herein. Generally, when lysates are compared, the numbers of cells and the amount of protein assayed may be determined, as well as, the use of identical expression systems and identical host cells to minimize variations in amount of enzyme produced by the host cells and present in the lysates.

The abbreviations used for the genetically encoded amino acids are conventional and are as follows:

TABLE 1
Abbreviations of amino acids

| Amino Acid | Three-Letter Abbreviation | One-Letter Abbreviation |
|---|---|---|
| Alanine | Ala | A |
| Arginine | Arg | R |
| Asparagine | Asn | N |
| Aspartate | Asp | D |
| Cysteine | Cys | C |
| Glutamate | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

### STATEMENT OF THE INVENTION

The present disclosure provides an aminotransaminase having aminotransaminase activity useful for the selective transamination of amino acceptor substrate compounds which in some embodiments, produce the pharmaceutical ingredient, (R)-3-aminobutan-1-ol (RABO). In an embodiment, the aminotransaminase has aminotransaminase an activity that is capable of converting the substrate compound to Formula (I). In a further embodiment, the amino acceptor substrate compound is 4-hydroxy-2-butanone. Further, the present disclosure provides nucleic acid sequences encoding the aminotransaminase of the present invention.

The aminotransaminase of the present disclosure is a non-naturally occurring aminotransaminase engineered to have improved enzyme properties, such as reaction yield and/or thermostability, as compared to the referenced aminotransaminase polypeptide of SEQ ID NO. 4.

According to the first aspect, the present invention provides a process for preparing a compound of structural Formula (I):

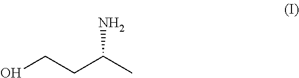

the process comprising the step of reacting 4-hydroxy-2-butanone with an amine donor in the presence of an aminotransaminase, wherein the aminotransaminase comprises an amino acid sequence that is at least 80% identical to SEQ ID NO. 4 and wherein the aminotransaminase comprises the following mutations relative to SEQ ID NO. 4: X5Q, X8A, X31Q, X54C, X61I, X94I, X102K, X136W, X162G, X181W, X187I, X199I, X209L and X223P.

According to a second aspect, the present invention provides an aminotransaminase comprising an amino acid sequence that is at least 80% identical to SEQ ID NO. 4 and wherein the aminotransaminase comprises the following mutations relative to SEQ ID NO. 4: X5Q, X8A, X31Q, X54C, X61I, X94I, X102K, X136W, X162G, X181W, X187I, X199I, X209L and X223P.

In one embodiment, the aminotransaminase of the present invention or as used in the process of the present invention additionally comprises the mutation X165N relative to SEQ ID NO. 4, i.e. the aminotransaminase comprises an amino acid sequence that is at least 80% identical to SEQ ID NO. 4 and comprises the following mutations relative to SEQ ID NO. 4: X5Q, X8A, X31Q, X54C, X61I, X94I, X102K, X136W, X162G, X181W, X187I, X199I, X209L, X223P and X165N.

In another embodiment, the aminotransaminase of the present invention or as used in the process of the present invention additionally comprises the mutation X215A relative to SEQ ID NO. 4, i.e. the aminotransaminase comprises an amino acid sequence that is at least 80% identical to SEQ ID NO. 4 and comprises the following mutations relative to SEQ ID NO. 4: X5Q, X8A, X31Q, X54C, X61I, X94I, X102K, X136W, X162G, X181W, X187I, X199I, X209L, X223P and X215N.

In one embodiment, the aminotransaminase of the present invention or as used in the process of the present invention comprises the following mutations relative to SEQ ID NO. 4: X5Q, X8A, X31Q, X54C, X61I, X94I, X102K, X136W, X162G, X181W, X187I, X199I, X209L, X223P, X165N and X215A.

In one embodiment, the aminotransaminase of the present invention or as used in the process of the present invention comprises an amino acid sequence that is at least 80%, 85%, 90% or 95% identical to SEQ ID NO. 4 and wherein the aminotransaminase comprises the following mutations relative to SEQ ID NO. 4: X5Q, X8A, X31Q, X54C, X61I, X94I, X102K, X136W, X162G, X181W, X187I, X199I, X209L and X223P.

In one embodiment, the aminotransaminase of the present invention or as used in the process of the present invention comprises an amino acid sequence that is at least 80%, 85%, 90% or 95% identical to SEQ ID NO. 4 and wherein the aminotransaminase comprises the following mutations relative to SEQ ID NO. 4: X5Q, X8A, X31Q, X54C, X61I, X94I, X102K, X136W, X162G, X181W, X187I, X199I, X209L, X223P and X165N.

In one embodiment, the aminotransaminase of the present invention or as used in the process of the present invention comprises an amino acid sequence that is at least 80%, 85%, 90% or 95% identical to SEQ ID NO. 4 and wherein the aminotransaminase comprises the following mutations relative to SEQ ID NO. 4: X5Q, X8A, X31Q, X54C, X61I, X94I, X102K, X136W, X162G, X181W, X187I, X199I, X209L, X223P and X215A.

In one embodiment, the aminotransaminase of the present invention or as used in the process of the present invention comprises an amino acid sequence that is at least 80%, 85%, 90% or 95% identical to SEQ ID NO. 4 and wherein the aminotransaminase comprises the following mutations relative to SEQ ID NO. 4: X5Q, X8A, X31Q, X54C, X61I, X94I, X102K, X136W, X162G, X181W, X187I, X199I, X209L, X223P, X165N and X215A.

In one embodiment, the aminotransaminase of the present invention or as used in the process of the present invention comprises the amino acid sequence of SEQ ID NO. 2. In another embodiment, the aminotransaminase of the present invention or as used in the process of the present invention consists of the amino acid sequence of SEQ ID NO. 2.

In one embodiment, the aminotransaminase of the present invention or as used in the process of the present invention provides an amine donor. In one embodiment, the amine donor is isopropylamine with the formula C₃H₉N. In one embodiment, the amine donor is a methylbenzylamine with the formula C₇H₉N. In an embodiment, the amine donor is R-methylbenzylamine with the formula C₆H₅CH(NH₂)CH₃.

In one embodiment, the aminotransaminase of the present invention or as used in the process of the present invention requires a cofactor. In one embodiment, the cofactor comprises a vitamin B₆ family compound. In one embodiment, the vitamin B₆ family compound is selected from the group consisting of PLP, PN, PL, PM, PYP, P5P, PNP and PMP. In one embodiment, the cofactor comprises PLP.

In one embodiment the present invention provides the process as set out in the first aspect of the invention or an aminotransaminase as set out in the second aspect of the invention, wherein the aminotransaminase comprises an amino acid sequence that is at least 80% identical to SEQ ID NO. 4 and wherein the aminotransaminase comprises the following mutations relative to SEQ ID NO. 4: X5Q, X8A, X31Q, X54C, X61I, X94I, X102K, X136W, X162G, X181W, X187I, X199I, X209L and X223P; and the aminotransaminase exhibits greater activity than the enzyme of SEQ ID NO. 4.

In one embodiment, the aminotransaminase of the present invention exhibits greater activity as measured using about 70 g/L, about 80 g/L, about 90 g/L or about 100 g/L 4-hydroxy-2-butanone, about 1.5M, about 1.75M, about 2.0M, about 2.25M or about 2.5M isopropylamine or about 1.0M, about 1.25, or about 1.5M R-methylbenzylamine, at about pH8, about pH9, about pH10 or about pH11, at about 30° C., about 35° C., about 37° C., about 40° C. to about 45° C., over about 20 hours, about 24 hours, about 28 hours, about 32 hours, about 36, about 40, about 44 or about 48 hours. In one embodiment, the aminotransaminase of the present invention exhibits greater activity relative to SEQ ID NO. 4 as measured using 100 g/L 4-hydroxy-2-butanone, 1.4M R-methylbenzylamine, at about pH8.0, about pH8.1, about pH8.2, about pH8.3, about pH8.4 or pH8.5, at about 30° C., about 31° C., about 32° C., about 33° C., about 34° C., about 35° C., about 36° C., about 37° C., about 38° C., about 39° C., about 40° C., about 41° C., about 42° C., about 43° C., about 44° C. or about 45° C., over about 20 hours. In one embodiment, the aminotransaminase of the present invention exhibits greater activity as measured using 100 g/L 4-hydroxy-2-butanone, 1.4M R-methylbenzylamine, at about pH8.5, at about 40° C., over about 20 hours.

In an embodiment, aminotransaminase of the present invention or as used in the process of the present invention has the transaminase activity of converting the substrate compound to Formula (I) with an activity that is increased at least about 300, 310, 320, 330, 340, 350, 400, 450, 500, 510, 520, 530, 540, 1000 FIOP or more relative to the activity of the reference polypeptide of SEQ ID NO. 4 under suitable reaction conditions (for example, as measured using 100 g/L 4-hydroxy-2-butanone, 1.4M R-methylbenzylamine, at about pH8.5, at 40° C.-45° C., over 24 hours). In an embodiment, the aminotransaminase of the present invention has a transaminase activity of converting the substrate compound to Formula (I) with an activity that is increased at least about 300 FIOP to 500 FIOP relative to the activity of the reference polypeptide of SEQ ID NO. 4. In an embodiment, the aminotransaminase has a transaminase activity of converting the substrate compound to Formula (I) with an activity that is increased at least about 350 FIOP to 550 FIOP relative to the activity of the reference polypeptide of SEQ ID NO. 4. In an embodiment, the aminotransaminase has a transaminase activity of converting the substrate compound to Formula (I) with an activity that is increased about 360 FIOP or about 540 FIOP.

Thus, in an embodiment, the present invention provides a process as set out in the first aspect of the invention, or an aminotransaminase as set out in the second aspect of the invention, wherein the aminotransaminase comprises an amino acid sequence that is at least 80% identical to SEQ ID NO. 4 and wherein the aminotransaminase comprises the following mutations relative to SEQ ID NO. 4: X5Q, X8A, X31Q, X54C, X61I, X94I, X102K, X136W, X162G, X181W, X187I, X199I, X209L and X223P; and the aminotransaminase exhibits an increased activity compared to the enzyme of SEQ ID NO. 4 as measured by a FIOP of greater than 300.

In an embodiment, the present invention provides the process as set out in the first aspect of the invention or an aminotransaminase as set out in the second aspect of the invention, wherein the aminotransaminase comprises an amino acid sequence that is at least 80% identical to SEQ ID NO. 4 and wherein the aminotransaminase comprises the following mutations relative to SEQ ID NO. 4: X5Q, X8A, X31Q, X54C, X61I, X94I, X102K, X136W, X162G, X181W, X187I, X199I, X209L, X223P, X165N and X215A; and the aminotransaminase exhibits an increased activity compared to the enzyme of SEQ ID NO. 4 as measured by a FIOP of greater than 500. In an embodiment, the aminotransaminase of the present invention has transaminase activity capable of converting the substrate compound to Formula (I) as described in Example 4 with a percent conversion of at least about 40%, at least about 50%, at least about 60%, at least about 70%, at least about 80%, at least about 90%, at least about 95%, at least 96%, at least 97%, at least about 98%, or at least about 99%, in a reaction time of about 48 h, about 36 h, about 24 h, about 20 h, or even a shorter length of time, under suitable reaction conditions (for example, as measured using 100 g/L 4-hydroxy-2-butanone, 1.4M R-methylbenzylamine, at about pH8.5, at 40° C.-45° C., over about 20 hours). In an embodiment, the aminotransaminase of the present invention is capable of converting the substrate compound to Formula (I) with a percent conversion of at least about 90% in a reaction time of about 20-48 hours, for example about 20 hours, about 24 hours, about 30 hours, about 35 hours, about 40 hours, about 45 hours or about 48 hours. In one embodiment, the percent conversion is at least about 95% in a reaction time of about 20-48 hours, for example about 20 hours, about 24 hours, about 30 hours, about 35 hours, about 40 hours, about 45 hours or about 48 hours. In one embodiment, the aminotransaminase has transaminase activity capable of converting the substrate compound to Formula (I) within enantiomeric excess of at least 90%, 95%, 96%, 97%, 98%, 99%, or greater, under suitable reaction conditions of about 30° C., about 31° C., about 32° C., about 33° C., about 34° C., about 35° C., about 36° C., about 37° C., about 38° C., about 39° C., about 40° C., about 41° C., about 42° C., about 43° C., about 44° C. or about 45° C., over about 20 hours, about 21 hours, about 22 hours, about 23 hours or about 24 hours, as measured using about 70 g/L, about 80 g/L, about 90 g/L or about 100 g/L 4-hydroxy-2-butanone, about 1.5M, about 1.75M, about 2.0M, about 2.25M or about 2.5M isopropylamine or about 1.0M, about 1.25, or about 1.5M R-methylbenzylamine, at about pH8, about pH9, about pH10 or about pH11. In one embodiment, the aminotransaminase has transaminase activity capable of converting the substrate compound to Formula (I) within enantiomeric excess of at least 90%, 95%, 96%, 97%, 98%, 99%, or greater, under suitable reaction conditions of about 30° C., about 31° C., about 32° C., about 33° C., about 34° C., about 35° C., about 36° C., about 37° C., about 38° C., about 39° C., about 40° C., about 41° C., about 42° C., about 43° C., about 44° C. or about 45° C., over about 20 hours, about 21 hours, about 22 hours, about 23 hours or about 24 hours, as measured using 100 g/L 4-hydroxy-2-butanone, 1.4M R-methylbenzylamine, at about pH8.5.

In one embodiment the present invention provides the process as set out in the first aspect of the invention or an aminotransaminase as set out in the second aspect of the invention, wherein the aminotransaminase comprises an amino acid sequence that is at least 80% identical to SEQ ID NO. 4 and wherein the aminotransaminase comprises the following mutations relative to SEQ ID NO. 4: X5Q, X8A, X31Q, X54C, X61I, X94I, X102K, X136W, X162G, X181W, X187I, X199I, X209L and X223P; and the aminotransaminase exhibits greater thermostability than the enzyme of SEQ ID NO. 4. In one embodiment, the aminotransaminase of the present invention exhibits increased activity at 50° C. than the enzyme of SEQ ID NO. 4.

The present invention also provides a composition comprising an aminotransaminase as defined in the second aspect of the invention.

In an embodiment, the aminotransaminase is an immobilized aminotransaminase. In an embodiment, the immobilized aminotransaminase is coupled to a protein immobilization bead.

The process of the present invention comprises reacting a butanone with an amine donor. Butanone, also known as methyl ethyl ketone, is an organic compound with the formula CH₃C(O)CH₂CH₃.

In an embodiment, the present invention provides a polynucleotide that encodes an aminotransaminase as set out in the first aspect of the invention, or an aminotransaminase as set out in the second aspect of the invention. In an embodiment, the polynucleotide comprises the nucleotide sequence of SEQ ID NO. 1. In an embodiment, the polynucleotide consists of the nucleotide sequence of SEQ ID NO. 1.

In an embodiment, the present invention provides the DNA sequence of polynucleotides that encodes an aminotransaminase as set out in the first aspect of the invention, or an aminotransaminase as set out in the second aspect of the invention. Where the sequence of the engineered polypeptide is known, the polynucleotides encoding the enzyme can be prepared by standard solid-phase methods, according to known synthetic methods. In some embodiments, fragments of up to about 100 bases can be individually synthesized, then joined (e.g., by enzymatic or chemical litigation methods, or polymerase mediated methods) to form any desired continuous sequence. For example, polynucleotides and oligonucleotides of the invention can be prepared by chemical synthesis using, e.g., the classical phosphoramidite method described by Beaucage, et al., 1981, Tet Lett 22:1859-69, or the method described by Matthes, et al., 1984, EMBO J. 3:801-05, e.g., as it is typically practiced in automated synthetic methods. According to the phosphoramidite method, oligonucleotides are synthesized, e.g., in an automatic DNA synthesizer, purified, annealed, ligated and cloned in appropriate vectors. In addition, essentially any nucleic acid can be obtained from any of a variety of commercial sources, The Great American Gene Company, Ramona, CA, ExpressGen Inc, Chicago, IL, Operon Technologies Inc, Alameda, CA and many others.

The following non-limiting Examples illustrate the present invention.

### EXAMPLES

### Example 1: Production of Aminotransferase Powders—Shake Flask Procedure

A single microbial colony of E. coli containing a plasmid encoding an aminotransferase of interest was inoculated into 50 mL Luria Bertoni broth containing 30 μg/mL chloramphenicol and 1% glucose. Cells were grown overnight (at least 16 hrs) in an incubator at 30° C. with shaking at 250 rpm. The culture was diluted into 1000 mL of 2×YT containing 30 μg/mL chloramphenicol (supplemented with 0.1 mM pyridoxine) to give an approximate OD600 of 0.2 and allowed to grow at 30° C. with shaking at 250 rpm. Expression of the aminotransferase gene was induced by the addition of isopropyl P D-thiogalactoside (IPTG) to a final concentration of 1 mM when the OD600 of the culture is 0.6 to 0.8. Incubation was then continued overnight (at least 16 hrs). Cells were harvested by centrifugation (3738 RCF, 20 min, 4° C.) and the supernatant was discarded. Pellets were frozen for 2 hours at −80° C. Pellets were then thawed and resuspended at a ratio of 4 mL 40 mM Potassium phosphate buffer (pH7.4) per gram of final pellet mass (e.g., 10 g frozen pellet suspended in 40 mL buffer). After resuspension, cells were filtered through 200 um mesh before passing twice through the microfluidizer at 12000 psig. Cell debris was removed by centrifugation (15,777 RCF, 40 min, 4° C.). The clear lysate supernatant was collected, pooled and lyophilized to provide a dry powder of crude aminotransferase enzyme.

### Example 2: Production of Aminotransferase Powders—Fermentation Procedure

An aliquot of frozen working stock (E. coli containing plasmid with the aminotransferase gene of interest) was removed from the freezer and allowed to thaw at room temperature. 300 μL of this working stock was inoculated into a primary seed stage of 250 ml M9YE broth (1.0 g/L ammonium chloride, 0.5 g/L of sodium chloride, 6.0 g/L of disodium monohydrogen phosphate, 3.0 g/L of potassium dihydrogen phosphate, 2.0 g/L of Procelys Springer 0251 yeast extract, 1 L/L de-ionized water) containing 30 μg/ml chloramphenicol and 1% glucose in 1 L flasks and allowed to grow at 37° C. with shaking at 200 rpm. When the OD600 of the culture was 0.5 to 1.0, the flasks were removed from the incubator and immediately used to inoculate a secondary seed stage.

A secondary seed stage was carried out in bench scale 5 L fermenters using 4 L of growth medium (0.88 g/L ammonium sulfate, 0.98 g/L of tri sodium citrate dihydrate; 12.5 g/L of dipotassium hydrogen phosphate, 6.25 g/L of potassium dihydrogen phosphate, 3.3 g/L of Procelys Springer 0251 yeast extract, 0.083 g/L ferric ammonium citrate, 0.5 ml/L polypropylene glycol antifoam and 8.3 ml/L of a trace element solution, 1 L/L process water. Trace element solution contained 2 g/L of calcium chloride dihydrate, 2.2 g/L of zinc sulfate heptahydrate, 0.5 g/L manganese sulfate monohydrate, 1 g/L copper sulfate pentahydrate, 0.1 g/L ammonium molybdate tetrahydrate and 0.02 g/L sodium tetraborate decahydrate, 1 L/L de-ionized water). Growth medium was sterilized at 121° C. for 40 minutes. Post sterilization 40 ml/L of feed stock solution was added (feed stock solution contained 12 g/L ammonium sulfate, 5.1 g/L magnesium sulfate heptahydrate, 500 g/L dextrose monohydrate, 1 L/L process water, sterilized at 121° C. for 30 minutes). Fermenters were inoculated with 2 ml OD600 0.5-1.0 primary seed and supplemented with 30 μg/ml chloramphenicol, incubated at 37° C., 300 rpm and 0.5vvm aeration. When the OD600 of the culture was 0.5-1.0 the secondary seed was immediately transferred to a final stage fermentation.

The final stage fermentation was carried out at bench scale in 10 L fermenters using 6 L of growth medium (0.88 g/L ammonium sulfate, 0.98 g/L of tri sodium citrate dihydrate; 12.5 g/L of dipotassium hydrogen phosphate, 6.25 g/L of potassium dihydrogen phosphate, 3.3 g/L of Procelys Springer 0251 yeast extract, 0.083 g/L ferric ammonium citrate, 0.5 ml/L polypropylene glycol antifoam and 8.3 ml/L of a trace element solution, 1 L/L process water. Trace element solution contained 2 g/L of calcium chloride dihydrate, 2.2 g/L of zinc sulfate heptahydrate, 0.5 g/L manganese sulfate monohydrate, 1 g/L copper sulfate pentahydrate, 0.1 g/L ammonium molybdate tetrahydrate and 0.02 g/L sodium tetraborate decahydrate, 1 L/L de-ionized water). Growth medium was sterilized at 121° C. for 40 minutes. Post sterilization growth medium was supplemented with 0.035 g/L pyridoxine hydrochloride and 40 ml/L feed stock solution (feed stock solution contained 12 g/L ammonium sulfate, 5.1 g/L magnesium sulfate heptahydrate, 500 g/L dextrose monohydrate, 1 L/L process water, sterilized at 121° C. for 30 minutes).

Fermenters were inoculated with 500 ml OD600 0.5-1.0 secondary seed and incubated at 37° C. and 1.5vvm aeration. Dissolved oxygen was controlled at 30% by variable speed agitation, pH was maintained at 7.0 by addition of 17.5% v/v ammonium hydroxide solution. Growth of the culture was maintained by addition of feed stock solution (12 g/L ammonium sulfate, 5.1 g/L magnesium sulfate heptahydrate, 500 g/L dextrose monohydrate, 1 L/L process water, sterilized at 121° C. for 30 minutes). After the culture reached an OD600 of 80+/−10 the temperature was reduced to 30° C. and isopropyl-β-D-thiogalactoside (IPTG) was added to a final concentration of 1 mM. The fermentation was continued for another 18 hours. At harvest, the culture was chilled to 8° C. Cells were collected by centrifugation at 5000 G for 40 minutes in a Sorvall RC12BP centrifuge at 4° C. Harvested cell pellets were then frozen at −80° C. and stored until downstream processing and recovery.

### Example 3: Production of Aminotransferase Catalyst—Immobilization Procedure

Protein content of lyophilized crude aminotransferase was quantified by Bradford Assay. 15 g Crude enzyme additions were calculated to a final protein concentration of 27 mg protein per gram resin (500-650 mg) and were weighed into a 50 mL conical flask. 15 mL resuspension buffer (1.14 g/L KH2PO4, 5.47 g/L K2HPO4 and 1.6 g/L PLP) was added and the protein suspension was gently mixed by inversion.

Purolite Epoxy methacrylate resin (ECR8206F/5730) was weighed into glass 125 mL Erlenmeyer flasks. 3M high salt immobilization buffer was prepared, such that the final concentration during immobilization is 1.8M. 121.09 g K2HPO4 and 25.91 g KH2PO4 were added to 250 mL water and dissolved with heat. Solution was left to cool and 22.5 mL was added to the 15 g resin in glass 125 mL Erlenmeyer flasks. The protein solution was then added to the resin and buffer, covered with parafilm and incubated at 25° C., 115 rpm, over the weekend.

Resin was decanted onto a square of 200 um mesh and washed with wash buffer (1.25 g/L K2HPO4 and 0.35 g/L KH2PO4) until filtrate ran clear through a Buchner funnel. Resin was blotted with a paper towel to dry and stored at 4° C. until use.

### Example 4: High Throughput Screening for Identification of Variants of the Arthrobacter Spp. Aminotransferase Capable of Converting Ketone Substrate to Amine

The gene encoding Arthorbacter spp aminotransferase (SEQ ID NO. 4), constructed as described in Example 2, was mutagenized using methods described below and the population of altered DNA molecules was used to transform a suitable E. coli host strain. Antibiotic resistant transformants were selected and processed to identify those expressing an aminotransferase with an improved ability to carry out the reaction (the substrate compound and Formula (I)) under desired reaction conditions. Cell selection, growth, induced expression of aminotransferase variant enzymes and collection of cell pellets were as described below.

Recombinant E. coli colonies carrying a gene encoding aminotransferase were picked using a Q-PIX molecular devices robotic colony picker (Genetix USA, Inc., Boston, MA) into 96-well shallow well microtiter plates containing in each well 180 μL LB Broth, 1% glucose and 30 μg/mL chloramphenicol (CAM). Cells were grown overnight at 30° C. and 85% humidity with shaking at 200 rpm. A 20 μL aliquot of this culture was then transferred into 96-deep well plates containing 380 μL 2×YT broth and 30 μg/mL CAM, supplemented with 100 μl pyridoxine. After incubation of the deep-well plates at 30° C. and 85% humidity with shaking at 250 rpm for 2-3 hrs, recombinant gene expression within the cultured cells was induced by the addition of IPTG to a final concentration of 1 mM. The plates were then incubated at 30° C. and 85% humidity with shaking at 250 rpm for 18 hrs. Cells were pelleted by centrifugation (3738 RCF, 10 min, 4° C.), resuspended in 200 μL lysis buffer and lysed by shaking at room temperature for 2 hours. The lysis buffer for early-stage engineered aminotransferases 25 mM Triethanolamine, pH 7.5, 1 mg/mL lysozyme, 500 μg/mL polymyxin B sulfate and 1 mM pyridoxal phosphate.

The lysis buffer for late-stage engineered aminotransferases consisted of 100 mM Potassium Phosphate, pH 8.0, 1 mg/mL lysozyme, 500 μg/mL polymyxin B sulfate and 1 mM pyridoxal phosphate (PLP). After sealing the plates with air-permeable nylon seals, they were shaken vigorously for 2 hours at room temperature. Cell debris was pelleted by centrifugation (3738 RCF, 10 min., 4° C.) and the clear supernatant was assayed directly or stored at 4° C. until use.

For screening of early-stage engineered aminotransferases, isopropylamine was used as the amine donor. For all plates, ketone, amine donor, cosolvent and 1 mM PLP were premixed as a reaction mix concentrate solution and diluted to final concentration by addition of lysate to start the reaction. Each well of a Costar deep well plate was charged with reaction mix concentrate, followed by the addition of the recovered lysate supernatant using a Biomek FX robotic instrument (Beckman Coulter, Fullerton, CA). Plates were heat-sealed with aluminum/polypropylene laminate heat seal tape at 165° C. for 4 seconds and incubated at 650 rpm (INFORS Thermotron), for 20 hours. The conditions for the mutants are summarised below. Reactions were prepared for analysis as described in Example 5.

SEQ ID NO. 4: 30% lysate, 77 g/L 4-hydroxy-2-butanone, 1.5M isopropylamine (pH10), 5% v/v methanol, 30° C.

SEQ ID NO. 6: 30% lysate, 100 g/L 4-hydroxy-2-butanone, 2.5M isopropylamine (pH10), 5% v/v isopropanol, 30° C.

SEQ ID NO. 8: 25% lysate, 130 g/L 4-hydroxy-2-butanone, 2.5M isopropylamine (pH10.75), 25% v/v DMSO, 45° C.

For screening of late-stage engineered aminotransferases (e.g. SEQ ID NOs 10, 12, 2), R-methylbenzylamine (R-MBA) was used as an amine donor. A pre-incubation stage with acetophenone by-product was included in some instances. For pre-incubation each well of a Costar deep well plate was charged with 18 μL 50% Acetophenone in DMSO, followed by the addition of 71.1 μL of the recovered lysate supernatant using a Biomek FX robotic instrument (Beckman Coulter, Fullerton, CA. Plates were heat-sealed with aluminum/polypropylene laminate heat seal tape at 165° C. for 4 seconds and incubated at 40° C., 850 rpm (INFORS Thermotron), for 20 hours.

For all plates, ketone and amine donor were premixed as a reaction mix concentrate solution and diluted to final concentration by addition to pre-incubated lysate to start the reaction. Each well of a Costar deep well plate was charged with reaction mix concentrate using a Biomek FX robotic instrument (Beckman Coulter, Fullerton, CA). Plates were heat-sealed with aluminum/polypropylene laminate heat seal tape at 165° C. for 4 seconds and incubated at 650 rpm (INFORS Thermotron), for 20 hours. The conditions for the mutants are summarised below. Reactions were prepared for analysis as described in Example 5.

SEQ ID NO. 10: 35.5% lysate, pre-incubation with acetophenone then 100 g/L 4-hydroxy-2-butanone, 1.4M R-MBA (pH8.5), 40° C.

SEQ ID NO. 12: 35.5% lysate, pre-incubation with acetophenone then 100 g/L 4-hydroxy-2-butanone, 1.4M R-MBA (pH8.5), 45° C.

SEQ ID NO. 2: 35.5% lysate, pre-incubation with acetophenone then 100 g/L 4-hydroxy-2-butanone, 1.4M R-MBA (pH8.5), 35-45° C.

For screening of late-stage engineered aminotransferases with temperature challenge, reactions progressed as follows. Challenging at higher temperatures allowed for identifying mutations that correlated to increased stability. Ketone and amine donor were premixed as a reaction mix concentrate solution and diluted to final concentration by addition of lysate to start the reaction. Each well of a Costar deep well plate was charged with reaction mix concentrate, followed by the addition of the recovered lysate supernatant using a Biomek FX robotic instrument (Beckman Coulter, Fullerton, CA). Plates were heat-sealed with aluminum/polypropylene laminate heat seal tape at 165° C. for 4 seconds and incubated at 650 rpm (INFORS Thermotron), for 20 hours. The conditions for the mutants are summarised below. Reactions were prepared for analysis as described in Example 5.

SEQ ID NO. 12: 44.55% lysate, 100 g/L 4-hydroxy-2-butanone, 1.4M R-MBA (pH8.5), 50° C. SEQ ID NO 12 has an FIOP of 1.428 over SEQ ID NO. 10 under the provided above test conditions.

The above reaction of an aminotransferase converting a substrate compound to an amine in the above Example 4 can be shown as follows:

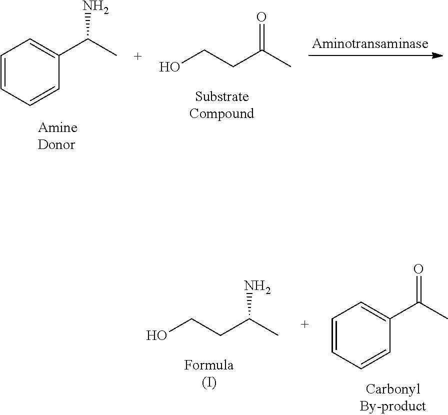

### Example 5: High Throughput Analytical Method for Identification of Variants of the Arthrobacter Spp. Aminotransferase Capable of Converting Ketone Substrate to Amine

### Derivatization of Amine Product by Marfey's Reagent:

Following overnight reaction, plates were removed from the incubator, seals removed and reactions were diluted with water. For early-stage engineered aminotransferases, samples were diluted 3-fold. For late-stage engineered aminotransferases, samples were diluted 10-fold (in two stages). Plates were heat-sealed with aluminum/polypropylene laminate heat seal tape at 165° C. for 4 seconds, shaken for 10 min and then centrifuged at 3738 RCF for 10 min to sediment debris.

1% Marfey's reagent (10 g/L in Acetonitrile) was prepared. 30 μL 1 M NaHCO3 was transferred to a new 96 deep-well plate, followed by 20 μL 10-fold diluted supernatant and 200 μL 1% Marfey's reagent. Plates were sealed and incubated at 40° C., 850 rpm (INFORS Thermotron), for 1 hour.

Derivatization was quenched by the addition of equal volume 1:9 2N HCl:Acetonitrile. Plates were mixed for 5 minutes then centrifuged at 3738 RCF for 10 min to sediment debris. Seals were removed and 20 μL supernatant was transferred to 180 μL acetonitrile in new shallow well polypropylene plates. Plates were again sealed, mixed and analyzed by UPLC.

UPLC method to qualitatively determine amine product following derivatization by Marfey's reagent: Enzymatic conversion of the ketone substrate of formula I (4-hydroxy-2-butanone commercially available, CAS number 590-90-9) to the amine of formula III was determined using an Agilent 1290 UPLC equipped with an Agilent Zorbax SB-C18 RRHD column (3.0×50 mm, 1.8 μm) using a gradient of 0.05% Trifluoroacetic Acid in Water (mobile phase A) and 0.05% Trifluoroacetic in Acetonitrile (mobile phase B) at a flow rate of 1.5 mL/min at a column temperature of 60° C. Beginning from a 75:25 ratio of A:B, the method followed a 0.2 minute hold, followed by a 0.4 minute gradient to 56:44 A:B, then a 0.15 minute purge gradient to 0:100 A:B, a 0.15 minute hold at 0:100 A:B and a 0.1 minute gradient to 80:20 A:B and finally a 0.25 minute hold at 80:20 A:B. Compound elution was monitored at 340, with excess Marfey's reagent eluting at 0.58 min, followed by derivatized RABO at 0.66 and excess amine donor (R-MBA) ketone eluting as a narrow peak, at 0.84 min.

TABLE 2
Fold Improvement over Parent (FIOP)

| | Single residue | Compounded FIOP |
|---|---|---|
| | mutations relative to | over enzyme of |
| Enzyme Sequence | SEQ ID NO. 4 | SEQ ID NO. 4 |
| SEQ ID NO 4 | — | 1.0 |
| SEQ ID NO 6 | 1 | 4.2 |
| SEQ ID NO 8 | 6 | 21.8 |
| SEQ ID NO 10 | 13 | 126.2 |
| SEQ ID NO 12 | 14 | 362.5 |
| SEQ ID NO 2 | 16 | 543.8 |

SEQUENCE LISTINGS

| | | |
|---|---|---|
| SEQ ID NO. | ATGGCTTTCTCTCAAGATACCGCGGAAATCGTTTACACCCA | |
| 1 | CGACACCGGTCTGGACTATATCACCTACTCTGACTACGAAC | |
| | TGGACCCGCAGAACCCGCTGGCTGGTGGTGCTGCTTGGAT | |
| | CGAAGGTGCTTTCGTTCCGCCGTCTGAAGCTCGTATCTGCA | |
| | TCTTCGACCAGGGTTACATTCACTCTGACGTTACCTACACC | |
| | GTTTTCCACGTTTGGAACGGTAACGCTTTCCGTCTGGACGA | |
| | CCACATCGAACGTCTGTTCTCTAACGCTGAATCTATTCGTA | |
| | TCATCCCGCCGCTGACCAAAGACGAAGTTAAAGAAATCGCT | |
| | CTGGAACTGGTTGCTAAAACCGAACTGCGTGAAGCATTCGT | |
| | TTCTGTTTCTATCACCCGTGGTTACTCTTCTACCCCATGGG | |
| | AACGTGACATCACCAAACATCGTCCGCAGGTTTACATGTAC | |
| | GCTGTTCCGTACCAGTGGATCGTACCGTTTGACGGCATCC | |
| | GTAACGGTGTTCACGCTATGGTTGCTCAGTCTGTTCGTCGT | |
| | ACTCCGCGTTGGTCTATCGACCCGCAGATTAAAAACTTCCA | |
| | GTGGGGTGACCTGATCCGTGCAATTCAGGAAACGCACGAC | |
| | CGTGGTTTCGAACTGCCGCTGCTGCTGGACGCAGACGGTT | |
| | TGCTGGCTGAAGGTCCAGGTTTCAACGTTGTTGTTATCAAA | |
| | GACGATGTTGTTCGTTCTCCGGGTCGTGCTGCTCTGCCGG | |
| | GTATCACCCGTAAAACCGTTCTGGAAATCGCTGAATCTCTG | |
| | GGTCACGAAGCTATCCTGGCTGACATCACCCTGGCTGAACT | |
| | GCTGGACGCTGACGAAGTTCTGGGTTGCACTACCGCTGGT | |
| | GGTGTTTGGCCGTTCGTTTCTGTTGACGGTAACCCGATCTC | |
| | TGACGGTGTTCCGGGTCCGGTTACCCAGTCTATCATCCGTC | |
| | GTTACTGGGAACTGAACGTTGAATCTTCTTCTCTGCTGACC | |
| | CCGGTACAGTACTAA | |
| SEQ ID NO. | MAFSQDTAEIVYTHDTGLDYITYSDYELDPQNPLAGGAAWI | Polypeptide of |
| 2 | EGAFVPPSEARICIFDQGYIHSDVTYTVFHVWNGNAFRLDD | SEQ ID NO. 1; |
| | HIERLFSNAESIRIIPPLTKDEVKEIALELVAKTELREAFVSVSI | Mutations of |
| | TRGYSSTPWERDITKHRPQVYMYAVPYQWIVPFDGIRNGV | SEQ ID NO. 121 |
| | HAMVAQSVRRTPRWSIDPQIKNFQWGDLIRAIQETHDRGF | D165N; |
| | ELPLLLDADGLLAEGPGFNVVVIKDDVVRSPGRAALPGITRK | G215A |
| | TVLEIAESLGHEAILADITLAELLDADEVLGCTTAGGVWPFVS | |
| | VDGNPISDGVPGPVTQSIIRRYWELNVESSSLLTPVQY | |
| SEQ ID NO. | ATGGCTTTCTCTGCTGACACCCCTGAAATCGTTTACACCCA | |
| 3 | CGACACCGGTCTGGACTATATCACCTACTCTGACTACGAAC | |
| | TGGACCCGGCTAACCCGCTGGCTGGTGGTGCTGCTTGGAT | |
| | CGAAGGTGCTTTCGTTCCGCCGTCTGAAGCTCGTATCTCTA | |
| | TCTTCGACCAGGGTTACCTGCACTCTGACGTTACCTACACC | |
| | GTTTTCCACGTTTGGAACGGTAACGCTTTCCGTCTGGACGA | |
| | CCACATCGAACGTCTGTTCTCTAACGCTGAATCTATGCGTA | |
| | TCATCCCGCCGCTGACCCAGGACGAAGTTAAAGAAATCGCT | |
| | CTGGAACTGGTTGCTAAAACCGAACTGCGTGAAGCATTCGT | |
| | TTCTGTTTCTATCACCCGTGGTTACTCTTCTACCCCATATG | |
| | AACGTGACATCACCAAACATCGTCCGCAGGTTTACATGTAC | |
| | GCTGTTCCGTACCAGTGGATCGTACCGTTTGACCGCATCCG | |
| | TGACGGTGTTCACGCTATGGTTGCTCAGTCTGTTCGTCGTA | |
| | CTCCGCGTTCTTCTATCGACCCGCAGGTTAAAAACTTCCAG | |
| | TGGGGTGACCTGATCCGTGCAGTTCAGGAAACCCACGACC | |
| | GTGGTTTCGAAGCTCCGCTGCTGCTGGACGGTGACGGTCT | |
| | GCTGGCTGAAGGTTCTGGTTTCAACGTTGTTGTTATCAAAG | |
| | ACGATGTTGTTCGTTCTCCGGGTCGTGCTGCTCTGCCGGG | |
| | TATCACCCGTAAAACCGTTCTGGAAATCGCTGAATCTCTGG | |
| | GTCACGAAGCTATCCTGGCTGACATCACCCTGGCTGAACTG | |
| | CTGGACGCTGACGAAGTTCTGGGTTGCACTACCGCTGGTG | |
| | GTGTTTGGCCGTTCGTTTCTGTTGACGGTAACCCGATCTCT | |
| | GACGGTGTTCCGGGTCCGGTTACCCAGTCTATCATCCGTC | |
| | GTTACTGGGAACTGAACGTTGAATCTTCTTCTCTGCTGACC | |
| | CCGGTACAGTACTAA | |
| SEQ ID NO. | MAFSADTPEIVYTHDTGLDYITYSDYELDPANPLAGGAAWIE | Polypeptide of |
| 4 | GAFVPPSEARISIFDQGYLHSDVTYTVFHVWNGNAFRLDDHI | SEQ ID NO. 3 |
| | ERLFSNAESMRIIPPLTQDEVKEIALELVAKTELREAFVSVSIT | |
| | RGYSSTPYERDITKHRPQVYMYAVPYQWIVPFDRIRDGVHA | |
| | MVAQSVRRTPRSSIDPQVKNFQWGDLIRAVQETHDRGFEAP | |
| | LLLDGDGLLAEGSGFNVVVIKDDVVRSPGRAALPGITRKTVLE | |
| | IAESLGHEAILADITLAELLDADEVLGCTTAGGVWPFVSVDGN | |
| | PISDGVPGPVTQSIIRRYWELNVESSSLLTPVQY | |
| SEQ ID NO. | ATGGCTTTCTCTGCTGACACCCCTGAAATCGTTTACACCCA | |
| 5 | CGACACCGGTCTGGACTATATCACCTACTCTGACTACGAAC | |
| | TGGACCCGGCTAACCCGCTGGCTGGTGGTGCTGCTTGGAT | |
| | CGAAGGTGCTTTCGTTCCGCCGTCTGAAGCTCGTATCTCTA | |
| | TCTTCGACCAGGGTTACCTGCACTCTGACGTTACCTACACC | |
| | GTTTTCCACGTTTGGAACGGTAACGCTTTCCGTCTGGACGA | |
| | CCACATCGAACGTCTGTTCTCTAACGCTGAATCTATGCGTA | |
| | TCATCCCGCCGCTGACCCAGGACGAAGTTAAAGAAATCGCT | |
| | CTGGAACTGGTTGCTAAAACCGAACTGCGTGAAGCATTCGT | |
| | TTCTGTTTCTATCACCCGTGGTTACTCTTCTACCCCATATG | |
| | AACGTGACATCACCAAACATCGTCCGCAGGTTTACATGTAC | |
| | GCTGTTCCGTACCAGTGGATCGTACCGTTTGACCGCATCCG | |
| | TGACGGTGTTCACGCTATGGTTGCTCAGTCTGTTCGTCGTA | |
| | CTCCGCGTTCTTCTATCGACCCGCAGGTTAAAAACTTCCAG | |
| | TGGGGTGACCTGATCCGTGCAGTTCAGGAAACCCACGACC | |
| | GTGGTTTCGAAGCTCCGCTGCTGCTGGACGGTGACGGTCT | |
| | GCTGGCTGAAGGTCCAGGTTTCAACGTTGTTGTTATCAAA | |
| | GACGATGTTGTTCGTTCTCCGGGTCGTGCTGCTCTGCCGG | |
| | GTATCACCCGTAAAACCGTTCTGGAAATCGCTGAATCTCTG | |
| | GGTCACGAAGCTATCCTGGCTGACATCACCCTGGCTGAACT | |
| | GCTGGACGCTGACGAAGTTCTGGGTTGCACTACCGCTGGT | |
| | GGTGTTTGGCCGTTCGTTTCTGTTGACGGTAACCCGATCTC | |
| | TGACGGTGTTCCGGGTCCGGTTACCCAGTCTATCATCCGTC | |
| | GTTACTGGGAACTGAACGTTGAATCTTCTTCTCTGCTGACC | |
| | CCGGTACAGTACTAA | |
| SEQ ID NO. | MAFSADTPEIVYTHDTGLDYITYSDYELDPANPLAGGAAWIE | Polypeptide of |
| 6 | GAFVPPSEARISIFDQGYLHSDVTYTVFHVWNGNAFRLDDHI | SEQ ID: 5; |
| | ERLFSNAESMRIIPPLTQDEVKEIALELVAKTELREAFVSVSIT | Mutations of |
| | RGYSSTPYERDITKHRPQVYMYAVPYQWIVPFDRIRDGVHA | SEQ ID NO. 4- |
| | MVAQSVRRTPRSSIDPQVKNFQWGDLIRAVQETHDRGFEAP | S223P |
| | LLLDGDGLLAEGPGFNVVVIKDDVVRSPGRAALPGITRKTVLE | |
| | IAESLGHEAILADITLAELLDADEVLGCTTAGGVWPFVSVDGN | |
| | PISDGVPGPVTQSIIRRYWELNVESSSLLTPVQY | |
| SEQ ID NO. | ATGGCTTTCTCTGCTGACACCCCTGAAATCGTTTACACCCA | |
| 7 | CGACACCGGTCTGGACTATATCACCTACTCTGACTACGAAC | |
| | TGGACCCGGCTAACCCGCTGGCTGGTGGTGCTGCTTGGAT | |
| | CGAAGGTGCTTTCGTTCCGCCGTCTGAAGCTCGTATCTCTA | |
| | TCTTCGACCAGGGTTACATTCACTCTGACGTTACCTACACC | |
| | GTTTTCCACGTTTGGAACGGTAACGCTTTCCGTCTGGACGA | |
| | CCACATCGAACGTCTGTTCTCTAACGCTGAATCTATTCGTA | |
| | TCATCCCGCCGCTGACCCAGGACGAAGTTAAAGAAATCGCT | |
| | CTGGAACTGGTTGCTAAAACCGAACTGCGTGAAGCATTCGT | |
| | TTCTGTTTCTATCACCCGTGGTTACTCTTCTACCCCATGGG | |
| | AACGTGACATCACCAAACATCGTCCGCAGGTTTACATGTAC | |
| | GCTGTTCCGTACCAGTGGATCGTACCGTTTGACCGCATCCG | |
| | TGACGGTGTTCACGCTATGGTTGCTCAGTCTGTTCGTCGTA | |
| | CTCCGCGTTCTTCTATCGACCCGCAGATTAAAAACTTCCAG | |
| | TGGGGTGACCTGATCCGTGCAGTTCAGGAAACCCACGACC | |
| | GTGGTTTCGAACTGCCGCTGCTGCTGGACGGTGACGGTCT | |
| | GCTGGCTGAAGGTCCAGGTTTCAACGTTGTTGTTATCAAA | |
| | GACGATGTTGTTCGTTCTCCGGGTCGTGCTGCTCTGCCGG | |
| | GTATCACCCGTAAAACCGTTCTGGAAATCGCTGAATCTCTG | |
| | GGTCACGAAGCTATCCTGGCTGACATCACCCTGGCTGAACT | |
| | GCTGGACGCTGACGAAGTTCTGGGTTGCACTACCGCTGGT | |
| | GGTGTTTGGCCGTTCGTTTCTGTTGACGGTAACCCGATCTC | |
| | TGACGGTGTTCCGGGTCCGGTTACCCAGTCTATCATCCGTC | |
| | GTTACTGGGAACTGAACGTTGAATCTTCTTCTCTGCTGACC | |
| | CCGGTACAGTACTAA | |
| SEQ ID NO. | MAFSADTPEIVYTHDTGLDYITYSDYELDPANPLAGGAAWIE | Polypeptide of |
| 8 | GAFVPPSEARISIFDQGYIHSDVTYTVFHVWNGNAFRLDDHI | SEQ ID: 7; |
| | ERLFSNAESIRIIPPLTQDEVKEIALELVAKTELREAFVSVSITR | Mutations of |
| | GYSSTPWERDITKHRPQVYMYAVPYQWIVPFDRIRDGVHA | SEQ ID NO. 6- |
| | MVAQSVRRTPRSSIDPQIKNFQWGDLIRAVQETHDRGFELP | L61I; M94I; |
| | LLLDGDGLLAEGPGFNVVVIKDDVVRSPGRAALPGITRKTVLE | Y136W; |
| | IAESLGHEAILADITLAELLDADEVLGCTTAGGVWPFVSVDGN | V187I; A209L |
| | PISDGVPGPVTQSIIRRYWELNVESSSLLTPVQY | |
| SEQ ID NO. | ATGGCTTTCTCTCAAGATACCGCGGAAATCGTTTACACCCA | |
| 9 | CGACACCGGTCTGGACTATATCACCTACTCTGACTACGAAC | |
| | TGGACCCGCAGAACCCGCTGGCTGGTGGTGCTGCTTGGAT | |
| | CGAAGGTGCTTTCGTTCCGCCGTCTGAAGCTCGTATCTGCA | |
| | TCTTCGACCAGGGTTACATTCACTCTGACGTTACCTACACC | |
| | GTTTTCCACGTTTGGAACGGTAACGCTTTCCGTCTGGACGA | |
| | CCACATCGAACGTCTGTTCTCTAACGCTGAATCTATTCGTA | |
| | TCATCCCGCCGCTGACCAAAGACGAAGTTAAAGAAATCGCT | |
| | CTGGAACTGGTTGCTAAAACCGAACTGCGTGAAGCATTCGT | |
| | TTCTGTTTCTATCACCCGTGGTTACTCTTCTACCCCATGGG | |
| | AACGTGACATCACCAAACATCGTCCGCAGGTTTACATGTAC | |
| | GCTGTTCCGTACCAGTGGATCGTACCGTTTGACGGCATCC | |
| | GTGACGGTGTTCACGCTATGGTTGCTCAGTCTGTTCGTCG | |
| | TACTCCGCGTTGGTCTATCGACCCGCAGATTAAAAACTTCC | |
| | AGTGGGGTGACCTGATCCGTGCAGTTCAGGAAACCCACGA | |
| | CCGTGGTTTCGAACTGCCGCTGCTGCTGGACGGTGACGGT | |
| | CTGCTGGCTGAAGGTCCAGGTTTCAACGTTGTTGTTATCAA | |
| | AGACGATGTTGTTCGTTCTCCGGGTCGTGCTGCTCTGCCG | |
| | GGTATCACCCGTAAAACCGTTCTGGAAATCGCTGAATCTCT | |
| | GGGTCACGAAGCTATCCTGGCTGACATCACCCTGGCTGAA | |
| | CTGCTGGACGCTGACGAAGTTCTGGGTTGCACTACCGCTG | |
| | GTGGTGTTTGGCCGTTCGTTTCTGTTGACGGTAACCCGAT | |
| | CTCTGACGGTGTTCCGGGTCCGGTTACCCAGTCTATCATCC | |
| | GTCGTTACTGGGAACTGAACGTTGAATCTTCTTCTCTGCTG | |
| | ACCCCGGTACAGTACTAA | |
| SEQ ID NO. | MAFSQDTAEIVYTHDTGLDYITYSDYELDPQNPLAGGAAWI | Polypeptide of |
| 10 | EGAFVPPSEARICIFDQGYIHSDVTYTVFHVWNGNAFRLDDH | SEQ ID NO. 9; |
| | IERLFSNAESIRIIPPLTKDEVKEIALELVAKTELREAFVSVSIT | Mutations of |
| | RGYSSTPWERDITKHRPQVYMYAVPYQWIVPFDGIRDGVHA | SEQ ID NO. 8- |
| | MVAQSVRRTPRWSIDPQIKNFQWGDLIRAVQETHDRGFELP | A5Q; P8A; |
| | LLLDGDGLLAEGPGFNVVVIKDDVVRSPGRAALPGITRKTVLE | A31Q; S54C; |
| | IAESLGHEAILADITLAELLDADEVLGCTTAGGVWPFVSVDGN | Q102K; |
| | PISDGVPGPVTQSIIRRYWELNVESSSLLTPVQY | R162G; S181W |
| SEQ ID NO. | ATGGCTTTCTCTCAAGATACCGCGGAAATCGTTTACACCCA | |
| 11 | CGACACCGGTCTGGACTATATCACCTACTCTGACTACGAAC | |
| | TGGACCCGCAGAACCCGCTGGCTGGTGGTGCTGCTTGGAT | |
| | CGAAGGTGCTTTCGTTCCGCCGTCTGAAGCTCGTATCTGCA | |
| | TCTTCGACCAGGGTTACATTCACTCTGACGTTACCTACACC | |
| | GTTTTCCACGTTTGGAACGGTAACGCTTTCCGTCTGGACGA | |
| | CCACATCGAACGTCTGTTCTCTAACGCTGAATCTATTCGTA | |
| | TCATCCCGCCGCTGACCAAAGACGAAGTTAAAGAAATCGCT | |
| | CTGGAACTGGTTGCTAAAACCGAACTGCGTGAAGCATTCGT | |
| | TTCTGTTTCTATCACCCGTGGTTACTCTTCTACCCCATGGG | |
| | AACGTGACATCACCAAACATCGTCCGCAGGTTTACATGTAC | |
| | GCTGTTCCGTACCAGTGGATCGTACCGTTTGACGGCATCC | |
| | GTGACGGTGTTCACGCTATGGTTGCTCAGTCTGTTCGTCG | |
| | TACTCCGCGTTGGTCTATCGACCCGCAGATTAAAAACTTCC | |
| | AGTGGGGTGACCTGATCCGTGCAATTCAGGAAACCCACGA | |
| | CCGTGGTTTCGAACTGCCGCTGCTGCTGGACGGTGACGGT | |
| | CTGCTGGCTGAAGGTCCAGGTTTCAACGTTGTTGTTATCAA | |
| | AGACGATGTTGTTCGTTCTCCGGGTCGTGCTGCTCTGCCG | |
| | GGTATCACCCGTAAAACCGTTCTGGAAATCGCTGAATCTCT | |
| | GGGTCACGAAGCTATCCTGGCTGACATCACCCTGGCTGAA | |
| | CTGCTGGACGCTGACGAAGTTCTGGGTTGCACTACCGCTG | |
| | GTGGTGTTTGGCCGTTCGTTTCTGTTGACGGTAACCCGAT | |
| | CTCTGACGGTGTTCCGGGTCCGGTTACCCAGTCTATCATCC | |
| | GTCGTTACTGGGAACTGAACGTTGAATCTTCTTCTCTGCTG | |
| | ACCCCGGTACAGTACTAA | |
| SEQ ID NO. | MAFSQDTAEIVYTHDTGLDYITYSDYELDPQNPLAGGAAWIE | Polypeptide of |
| 12 | GAFVPPSEARICIFDQGYIHSDVTYTVFHVWNGNAFRLDDHI | SEQ ID: 11; |
| | ERLFSNAESIRIIPPLTKDEVKEIALELVAKTELREAFVSVSITR | Mutations of |
| | GYSSTPWERDITKHRPQVYMYAVPYQWIVPFDGIRDGVHAM | SEQ ID NO. |
| | VAQSVRRTPRWSIDPQIKNFQWGDLIRAIQETHDRGFELPLL | 10-V199I |
| | LDGDGLLAEGPGFNVVVIKDDVVRSPGRAALPGITRKTVLEIA | |
| | ESLGHEAILADITLAELLDADEVLGCTTAGGVWPFVSVDGNPI | |
| | SDGVPGPVTQSIIRRYWELNVESSSLLTPVQY | |

1. A process for preparing a compound of Formula (I): 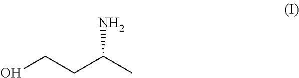
the process comprising the step of reacting 4-hydroxy-2-butanone with an amine donor in the presence of an aminotransaminase, wherein the aminotransaminase comprises an amino acid sequence that is at least 80% identical to SEQ ID NO. 4 and wherein the aminotransaminase comprises the following mutations relative to SEQ ID NO. 4: X5Q, X8A, X31Q, X54C, X61I, X94I, X102K, X136W, X162G, X181W, X187I, X199I, X209L and X223P.

2. The process as claimed in claim 1, wherein the aminotransaminase additionally comprises the mutation X165N relative to SEQ ID NO. 4.

3. The process as claimed in claim 1, wherein the aminotransaminase additionally comprises the mutation X215A relative to SEQ ID NO. 4.

4. The process as claimed in claim 1, wherein the aminotransaminase comprises an amino acid sequence that is at least 80%, 85%, 90% or 95% identical to SEQ ID NO. 4.

5. The process of claim 1, wherein the aminotransaminase comprises the amino acid sequence of SEQ ID NO. 2.

6. The process of claim 5 wherein the aminotransaminase consists of the amino acid sequence of SEQ ID NO. 2.

7. The process of claim 1, wherein the amine donor is a methylbenzylamine.

8. The process of claim 7, wherein the amine donor is R-methylbenzylamine.

9. An aminotransaminase comprising an amino acid sequence that is at least 80% identical to SEQ ID NO. 4 and wherein the aminotransaminase comprises the following mutations relative to SEQ ID NO. 4: X5Q, X8A, X31Q, X54C, X61I, X94I, X102K, X136W, X162G, X181W, X187I, X199I, X209L and X223P.

10. The aminotransaminase claimed in claim 9 which additionally comprises the following mutations relative to SEQ ID NO.4: X165N and X215A.

11. An aminotransaminase comprising an amino acid sequence of SEQ ID NO. 2.

12. An aminotransaminase consisting of the amino acid sequence of SEQ ID NO. 2.

13. A polynucleotide encoding an aminotransaminase as claimed in claim 9.

14. The polynucleotide as claimed in claim 13, wherein the polynucleotide comprises the nucleotide sequence of SEQ ID NO.1.

15. Use of an aminotransaminase as defined in claim 9, for the manufacture of a compound of Formula (I): [chemical structure]
